(19) United States
(12) Patent Application Publication
(10) Pub. No.: US 2025/0152420 A1
(43) Pub. Date: May 15, 2025

(54) SURGICAL KIT AND METHOD FOR TREATING GLAUCOMA

(71) Applicant: LIQID MEDICAL PROPRIETARY LIMITED, Cape Town, Western Cape Province (ZA)

(72) Inventors: Daemon Bruce McCLUNAN, Cape Town (ZA); Joshua David FISCHER, Cloetesville (ZA)

(21) Appl. No.: 18/728,657

(22) PCT Filed: Jan. 12, 2023
(86) PCT No.: PCT/IB2023/050286

§ 371 Date: Jul. 12, 2024

(30) Foreign Application Priority Data
Jan. 14, 2022 (ZA) 2022/00673

(51) Int. Cl. A61F 9/007; A61F 9/013

(52) U.S. Cl. CPC A61F 9/00781; A61F 9/0133

(57) ABSTRACT

A surgical kit includes a surgical instrument for creating a passageway though scleral tissue and a shunt locatable in the passageway for diverting aqueous fluid from an eye chamber. The shunt includes an elongate duct defining a fluid passageway for diverting aqueous humor from the chamber and a fixation body located on the duct for fixing the duct within scleral tissue. The fixation body defines an internal passage within which the duct is received. The surgical instrument has a flat cutting blade having a first maximal cross-sectional dimension which is larger than a second maximal cross-sectional dimension of the duct, which is less than a third maximal cross-sectional dimension of the fixation body. The flat cutting blade is minimally invasive, reducing the risk of aqueous fluid leakage and allowing the use of a fixation body having a relatively small cross-sectional dimension.

### TECHNICAL FIELD

This invention relates to a surgical kit for treating glaucoma in a patient. The invention relates also to a method for treating glaucoma in a patient. More specifically, the invention relates to a surgical kit and method for treating glaucoma in a patient by diverting aqueous fluid from a chamber of the eye to the subconjunctival space of the patient.

### BACKGROUND TO INVENTION

Glaucoma is an ocular disease characterised by the presence of raised intraocular pressure (IOP) causing irreversible damage to the optic nerve. The ocular globe of the eye has a tough outer layer comprised of the sclera and the cornea. The internal areas of the eye are separated into the anterior segment and the posterior segment. The anterior segment comprises the anterior and posterior chambers of the eye filled with aqueous fluid, and the posterior segment comprises the vitreous chamber filled with vitreous gel. The cornea merges into the sclera at a juncture referred to as the limbus. The limbus comprises an approximately 2 mm annular region surrounding the cornea, which includes a 1 mm so-called “blue zone” adjacent the cornea and a 1 mm so-called “white zone” adjacent the sclera. The trabecular meshwork lies under the limbus within the anterior chamber angle. A portion of the sclera is covered by a thin tissue called Tenon's membrane (also called Tenon's capsule), which envelopes the bulb of the eye from the optic nerve to the ciliary region. Near its front, Tenon's membrane blends into the conjunctiva where it is attached to the ciliary region of the eye.

The ocular globe maintains an internal pressure known as the intraocular pressure which normally varies between 10 mmHg and 21 mmHg. The intraocular pressure needs to be controlled within a defined range in order for the eye to function normally. The intraocular pressure is regulated by maintaining a balance between volumes of aqueous fluid produced and drained from the anterior segment of the ocular globe. Aqueous fluid is produced at a rate of about 2 to 3 micromillimetres per minute by the ciliary body and drained through the trabecular meshwork and uveoscleral pathways at variable rates. If an impairment occurs in the amount of aqueous fluid drained from the ocular globe, then the intraocular pressure becomes too high. The presence of raised intraocular pressure results in a large pressure differential across the lamina cribrosa (translaminar pressure). This causes damage to the optic nerve head known as glaucoma. Glaucoma causes irreversible visual field defects. These defects enlarge until a patient's field of view is severely restricted. In the end stage of the disease, total vision loss occurs. Glaucoma is a leading cause of blindness worldwide. If the intraocular pressure remains very high, the eye can become persistently painful and may need to be removed.

Current medical, laser and surgical treatment options for glaucoma are aimed at lowering intraocular pressure. Glaucoma which is difficult to control through first line therapies such as topical medications and laser therapy is known as refractory glaucoma. Refractory glaucoma is often managed by glaucoma drainage device implantation to create an additional aqueous outflow pathway into the subconjunctival space.

The general technique for subconjunctival glaucoma drainage device implantation includes the following steps:
A sub-tenons pocket is created by releasing the conjunctiva and tenon's membrane from their insertion near the limbus;
A channel which communicates the sub-tenons pocket with a chamber of the eye is created through the sclera at a depth of around 50% of the thickness of the sclera;
A drainage tube is passed along the scleral channel to create a fluid communication between the chamber of the eye and the subtenons/subconjunctival space;
The subtenons pocket is closed.

Aqueous fluid draining through the tube into the subconjunctival space creates a fluid blister between the sclera and the tenons/conjunctiva known as a bleb.

It is preferable for the scleral channel created to have the smallest dimension achievable to reduce the invasiveness of the procedure. The initial angle of the scleral channel needs to be sufficiently parallel to the ocular globe so as to pass along the sclera without prematurely penetrating the eye, however the final angle at which the scleral channel enters the anterior chamber needs to be approximately in the plane which bisects the angle between the iris and the cornea so that the tube entering the eye does not touch the corneal endothelium or iris tissue which could cause corneal endothelial decompensation or tube blockage respectively. Additionally, the point at which the scleral channel enters the anterior chamber needs to be at the region of the trabecular meshwork. Entering anterior to this may result in the tube damaging corneal endothelial stem cells and entering posterior to this may result in bleeding from the ciliary body. In the early stages following surgery, fluid leakage may occur between the tube and the scleral channel. Fluid leakage may result in IOP dropping below 5 mmHg which causes a condition known as hypotony. Hypotony may cause complications such as maculopathy and choroidal effusion. In the later stages, glaucoma devices may tend to erode through surrounding tissues which creates a risk for device migration and exposure. One factor which determines the likelihood for erosion of a device is the maximal dimension of the device. A glaucoma device implanted within a scleral channel needs to be sufficiently small so as to reduce invasiveness and the risk of erosion, but also sufficiently large so as to create an effective fluid seal within the scleral channel.

The MicroShunt kit (described in U.S. Pat. No. 9,101,444) comprises a surgical kit for treating elevated ocular pressure, including a surgical instrument having a needle body used to form a surgical passage through ocular tissue and a shunt defining a duct, for diverting aqueous fluid from the anterior chamber of the eye of a patient. The shunt consists of a straight tube of about 8 mm in length with a pair of sealing tabs extending in a common plane radially outwardly. A scleral channel is created from approximately 3 mm from the limbus radially along the sclera until the anterior chamber is entered using the needle body. The distal end of the tube is passed through the scleral channel to enter the anterior chamber. The sealing tabs are positioned in the scleral channel to create a fluid seal between the tube and surrounding scleral tissue. The proximal end is then left lying in the subconjunctival space to create a bleb. The sealing tabs have a maximal cross-sectional dimension that is greater than a maximal cross-sectional dimension of the needle body.

The design of the MicroShunt kit results in sealing tabs with a larger cross-sectional dimension than the instrument used to create the scleral channel. This increases the maximal cross-sectional dimension of the device and therefore the invasiveness and risk of erosion and migration. The kit furthermore includes the use of a needle body for creating a scleral channel. Needle bodies are known to be cumbersome when used during the creation of a scleral channel. This is due to the single bevelled tip and round body. The single bevel results in the instrument tending to track in the direction of the bevel when creating a channel. This creates difficulty for the surgeon when trying to maintain an accurate channel depth within the thin scleral wall. Additionally, the round body of the needle creates a round channel within the sclera which is more prone to aqueous fluid leakage.

It is an object of the present invention to provide a surgical kit and a method for treating glaucoma which addresses the abovementioned shortcomings of existing ocular shunt devices and kits for use in the treatment of glaucoma.

In this specification, the term “distal” means in the direction of the eye of a patient or away from a user of the shunt of the surgical kit, while the term “proximal” means in the direction away from the eye of the patient or towards the user of the shunt.

### SUMMARY OF INVENTION

According to a first aspect of the invention there is provided a surgical kit for treating glaucoma in a patient, the surgical kit comprising:

a surgical instrument having a flat cutting blade for creating a passageway through scleral tissue, the flat blade having a first maximal cross-sectional dimension;
a shunt for diverting aqueous humor from a chamber of the eye of the patient, the shunt comprising a deformable elongate duct having a distal portion which is locatable in the scleral passageway and which defines a distal end of the elongate duct; and a fixation body located on the elongate duct intermediate the ends of the elongate duct, the distal portion of the elongate duct of the shunt having a second maximal cross-sectional dimension that is less than the first maximal cross-sectional dimension; and the fixation body having a third maximal cross-sectional dimension that is greater than the second maximal cross-sectional dimension and less than the first maximal cross-sectional dimension.

The cutting blade of the surgical instrument may have bevelled cutting surfaces at opposite sides of the cutting blade at a tip region of the cutting blade. The bevelled cutting tip may have a length of approximately 2 mm.

The elongate duct of the shunt may have a proximal portion defining a proximal end of the elongate duct, the proximal portion being locatable externally of the scleral passageway.

The distal portion of the elongate duct may have a distal lumen defining a distal part of the fluid passageway and the proximal portion may have a proximal capillary lumen which is in fluid flow communication with the distal lumen, the proximal lumen having an internal diameter which is relatively smaller than an internal diameter of the distal lumen so as to reduce a flow rate of aqueous humor and regulate pressure along the proximal capillary lumen sufficient to prevent hypotony.

The chamber of the eye in which the shunt is implanted may be the anterior chamber, the posterior chamber or the vitreous chamber of the eye.

In a particular embodiment of the invention, the proximal portion of the elongate duct may be releasably connected to the distal portion of the elongate duct. In another embodiment of the invention, the proximal portion of the elongate duct may be fixedly connected to the distal portion of elongate duct.

The fixation body of the shunt may be slidably located on the distal portion of the elongate duct for fixing the distal portion of the duct within the scleral channel at a desired position determined by a required length of the shunt.

The fixation body may be frictionally located on the distal portion of the elongate duct in an arrangement wherein a coefficient of friction acting between the fixation body and the elongate duct is sufficient to adequately resist movement of the fixation body relative to the elongate duct when the elongate duct is implanted in the scleral passageway of the patient yet permits sliding displacement of the fixation body relative to the elongate duct when a moderate force is applied to the fixation body by a surgeon.

The fixation body may define an internal passage within which the elongate duct is received. The fixation body may be located on the elongate duct in an interference fit wherein an internal diameter of the fixation body is slightly less than an external diameter of the distal portion of the elongate duct.

The fixation body may have a pair of laterally-extending flanges which project outwardly from opposite sides thereof.

The fixation body may have a convexly rounded lower surface and a substantially flat upper surface.

The fixation body may be of a rigid construction.

The fixation body may define a curved internal passage in which the elongate duct is received. The curvature of the internal passage causes bending of the elongate duct received therein, thereby directing the distal end away from the corneal endothelium and towards the iris plane of the patient.

According to a second aspect of the invention there is provided a surgical method for treating glaucoma in a patient, the surgical method comprising:
providing a surgical kit as described and defined in accordance with the first aspect of the invention;
opening the conjunctival/tenon's complex to create a pocket between the conjunctival/tenon's complex and the sclera;
using the cutting blade of the surgical instrument, creating a passageway through scleral tissue so as to form a scleral channel from an external position at the pocket in the conjunctival/tenon's complex to a chamber of the eye from which aqueous fluid is to be diverted;
inserting the distal portion of the elongate duct of the shunt into the scleral channel until the distal end of the elongate duct lies within the chamber of the eye and the fixation body is located within the scleral channel for fixing and sealing the distal portion of the elongate duct within the scleral tissue at a desired position relative to the limbus; and
closing the conjunctival/tenon's complex leaving the proximal end of the shunt lying within the pocket.

The surgical method may include, prior to creating the passageway using the cutting blade, marking a point on the sclera in the pocket between the conjunctival/tenon's complex and the sclera at a predetermined distance from the limbus which may be approximately 3 mm from the limbus.

The surgical method may include creating the passageway through scleral tissue, from the predetermined position on the sclera to enter the chamber of the eye in a single step.

Alternatively, the surgical method may include creating the passageway through scleral tissue, from the predetermined position on the sclera to enter the chamber of the eye in three steps as follows:
1) using the cutting blade of the surgical instrument, creating a passageway through scleral tissue at an angle approximately parallel to the wall of the ocular globe so as to form a scleral channel extending from the predetermined position on the sclera to a point about 1 mm from the limbus;
2) changing the direction of the cutting blade towards the iris plane; and
3) advancing the cutting blade to enter the anterior chamber of the eye at about the region of the trabecular meshwork.

### BRIEF DESCRIPTION OF THE DRAWINGS

Further features of the invention are described in more detail hereinafter, by way of a non-limiting example of the invention, with reference to and as illustrated in the accompanying diagrammatic drawings. In the drawings:

FIG. 1 shows a prior art Illustration providing anatomic details of a human eye;

FIG. 2 shows a side view of a shunt of the surgical kit for treating glaucoma, in accordance with the invention, prior to cutting the shunt to a desired length;

FIG. 3 shows top plan view of the shunt of FIG. 2;

FIG. 4 shows a distal end view of the shunt of FIG. 2;

FIG. 5 shows a proximal end view of the shunt of FIG. 2;

FIG. 6 shows a perspective view as seen from above, of the fixation body of the shunt of FIG. 2;

FIG. 7 shows an end view of the fixation body;

FIG. 8 shows a sectional side view of the fixation body;

FIG. 9 shows a surgical instrument having a cutting blade, of the surgical kit in accordance with the invention;

FIG. 10 shows a side view of the surgical instrument of FIG. 9;

FIG. 11 provides a comparative illustration of the cross-sectional dimensions DMAX1 of the surgical blade and DMAX3 of the fixation body;

FIG. 12 provides a comparative illustration of the cross-sectional dimension DMAX3 and circumference C3 of the scleral channel deformed by the fixation body and the cross-sectional dimension DMAX1 and circumference C1 of the scleral channel formed by the surgical blade of FIG. 9;

FIG. 13 shows a side view of the shunt of FIG. 2, illustrating the manner in which the fixation body is displaced along the distal portion of the elongate duct using forceps;

FIG. 14 shows a side view of the shunt of FIG. 2, illustrating the manner in which the distal portion of the elongate duct is cut by a surgeon;

FIG. 15 shows a side view of the shunt of FIG. 2 after it has been cut to a desired length;

FIG. 16 shows a sectional side view of the shunt of FIG. 15;

FIG. 17 shows enlarged detail A of FIG. 16;

FIG. 18 illustrates the manner in which a channel is created in the sclera in one step using the surgical blade of FIG. 9 for insertion of the shunt of FIG. 2 into the anterior chamber of the eye of the patient;

FIG. 19 illustrates the shunt of FIG. 2 wherein the distal portion is cut so as to provide a relatively short section of tubing and implanted in the anterior chamber of the patient's eye and creating a bleb near to the limbus;

FIG. 20 illustrates the manner in which a channel is created in the sclera in one step using the surgical blade of FIG. 9 for insertion of the shunt of FIG. 2 into the posterior chamber of the eye of the patient;

FIG. 21 shows the implanted shunt of FIG. 2, implanted in the posterior chamber of the patient's eye;

FIG. 22 illustrates the manner in which a channel is created in the sclera in one step using the surgical blade of FIG. 9 for insertion of the shunt of FIG. 2 into the vitreous chamber of the eye of the patient;

FIG. 23 shows the implanted shunt of FIG. 2, implanted in the vitreous chamber of the patient's eye;

FIG. 24 illustrates a first step in the three-step process for forming a scleral channel in scleral tissue using a cutting blade;

FIG. 25 shows second step in the three-step process for forming a scleral channel in scleral tissue using a cutting blade; and

FIG. 26 shows third step in the three-step process for forming a scleral channel in scleral tissue using a cutting blade.

### DETAILED DESCRIPTION OF THE DRAWINGS

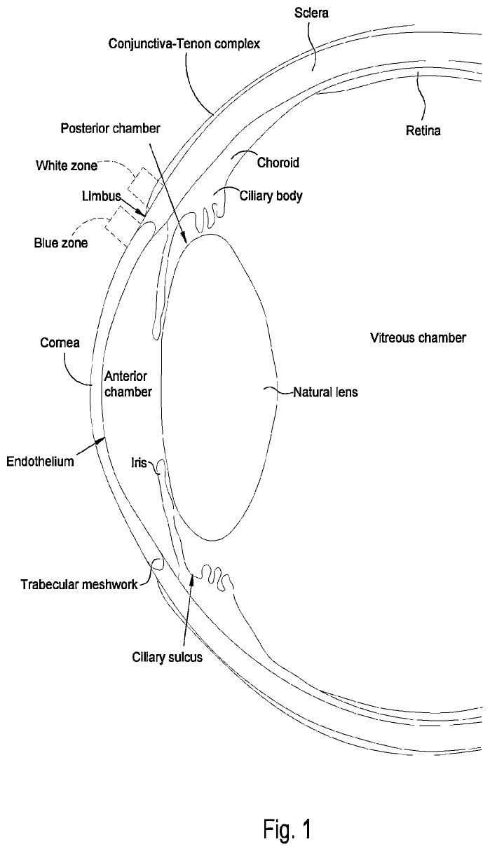

A surgical kit for treating glaucoma by lowering intraocular pressure in an eye of a patient, is described and illustrated. Anatomic details of a human eye are illustrated in FIG. 1.

The surgical kit comprises, broadly, a surgical instrument 50 for creating a passageway though scleral tissue and a shunt 10 which can be located in the passageway for diverting aqueous fluid from a chamber of the eye.

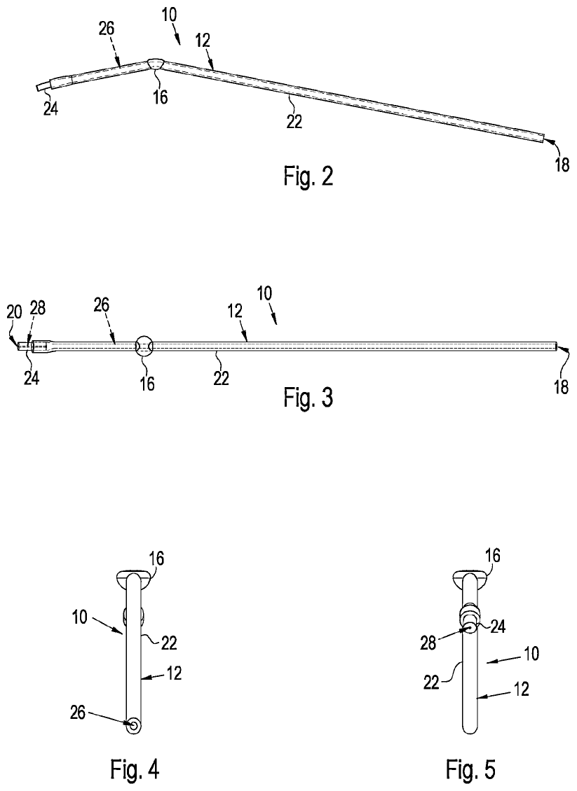

With reference to FIGS. 2-8, the shunt 10 comprises, broadly, an elongate silicone rubber duct 12 for defining a fluid passageway 14 for diverting aqueous humor from a chamber of the patient's eye and a fixation body 16 located on and extending outwardly from the duct 12 for fixing the duct within tissue surrounding the eye. The relevant chamber of the eye may be the anterior chamber or the posterior chamber of the eye.

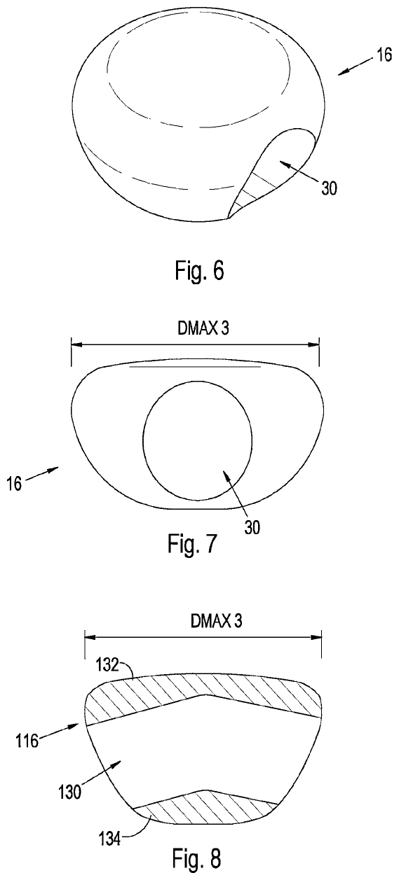

The elongate duct 12 has a distal end 18 and an opposite proximal end 20, the distal end 18 being implantable in the relevant chamber of the eye. The elongate duct has a distal portion 22 defining the distal end of the duct, which is locatable in the scleral channel of the patient, the distal portion being deformable so as to permit the distal portion to conform to anatomical structures of the eye of the patient. The distal portion is also severable, allowing a surgeon to cut the distal portion to a desired length corresponding to the anatomical dimensions of the patient's eye and required bleb position. As is illustrated in FIGS. 6-8, a bevel cut is made in the distal portion so as to define a relatively sharp point at a distal end 18.1 for facilitating insertion of the distal portion along a channel defined in scleral tissue.

The elongate duct 12 further has a proximal portion 24 having a rigid construction, which defines the proximal end of the elongate duct.

The distal portion 22 has a distal lumen 26 defining a distal part of the fluid passageway and the proximal portion 24 has a proximal capillary lumen 28 which is in fluid flow communication with the distal lumen, the proximal lumen having an internal diameter which is relatively smaller than an internal diameter of the distal lumen so as to reduce a flow rate of aqueous humor along the proximal capillary lumen.

The shunt is configured to resist aqueous fluid flow at flow rates of between 1.5 to 3.0micromillimetres per minute through the shunt using the Haigen Pouseille equation and a viscosity factor of 7.042 cP and more specifically, about 2 micromillimetres per minute.

The distal lumen has a diameter of between 0.12 mm and 0.3 mm.

The proximal capillary lumen has a diameter of between 0.035 mm and 0.06 mm.

The distal portion has a length of between 4 mm and 30 mm after cutting by the surgeon.

The proximal portion has a length of between 1 mm and 8 mm. The distal portion is configured to provide negligible fluid flow resistance less than 1 mmHg while the proximal capillary portion is configured to provide significant fluid flow resistance of between 4 mmHg and 12 mmHg.

In a particular embodiment of the invention, the proximal portion of the elongate duct is releasably connected to the distal portion of the elongate duct. In another embodiment of the invention, the proximal portion of the elongate duct is fixedly connected to the distal portion of elongate duct.

The proximal portion of the elongate duct has an oval shape when viewed in cross section. The proximal portion has a curvature that conforms to that of an anatomical curvature of the ocular globe.

The fixation body 14 of the shunt forms an effective seal with surrounding scleral tissue. The fixation body is slidably located on the distal portion of the elongate duct 12 for fixing the distal portion 22 of the duct within a channel created in scleral tissue at a desired position determined by a required length of the shunt as will be explained in more detail hereinbelow.

The fixation body 14 is frictionally located on the distal portion 22 of the elongate duct in an arrangement wherein a coefficient of friction acting between the fixation body and the elongate duct is sufficient to adequately resist movement of the fixation body relative to the elongate duct following implantation yet permit sliding displacement of the fixation body relative to the elongate duct when a force is applied to the fixation body by a surgeon during implantation. The elongate duct is deformed slightly inwardly in the region where it is engaged by the fixation body.

The fixation body defines a curved internal passage 30 within which the distal portion of the elongate duct is received. The internal passage 30 of the fixation body and the distal portion of the elongate duct are cylindrical. The curvature of the internal passage 30 is configured so as to cause bending of the elongate duct when the elongate duct is received therein, thereby to resist displacement of the shunt within a scleral channel created within scleral tissue and direct the distal end away from the corneal endothelium and towards the iris plane of the patient.

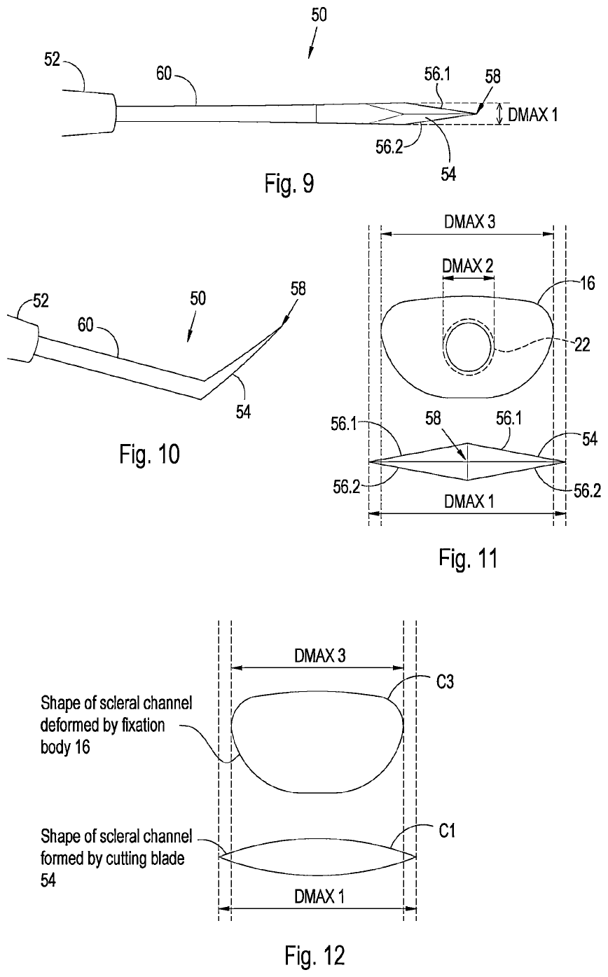

With reference to FIGS. 9 and 10, a side view of the surgical instrument 50 is illustrated. The surgical instrument includes a handle 52 and a flat cutting blade 54 having bevelled cutting surfaces 56.1 and 56.2 at opposite sides of the cutting blade near a tip 58 of the cutting blade. The cutting blade 54 is disposed at an angle relative to a shank 60 of the surgical instrument. The flat cutting blade has a first maximal cross-sectional dimension DMAX1.

The distal portion 22 of the elongate duct 12 has a second maximal cross-sectional dimension DMAX2 which is less than the first maximal cross-sectional dimension DMAX1.

The fixation body 14 has a third maximal cross-sectional dimension DMAX3 which is greater than the second maximal cross-sectional dimension DMAX2 but less than the first maximal cross-sectional dimension DMAX1.

The use of a flat cutting blade for creating the passageway in the scleral tissue for the elongate duct of the shunt, is minimally invasive, reduces the risk of aqueous fluid leakage and allows the use of a fixation body having a relatively small cross-sectional dimension DMAX3 which is less than the width of the cutting blade (DMAX1).

FIG. 11 shows a comparative illustration of the cross-sectional dimensions DMAX1 of the surgical blade and DMAX3 of the fixation body.

FIG. 12 shows a comparative illustration of the cross-sectional dimension DMAX3 and circumference C3 of the scleral channel deformed by the fixation body and the cross-sectional dimension DMAX1 and circumference C1 of the scleral channel formed by the surgical blade. Scleral tissue deforms as it is stretched around the fixation body forming a seal around the fixation body. The circumference C3 of the fixation body is larger than the circumference C1 of the surgical blade allowing scleral tissue to stretch around the fixation body forming the seal mentioned above.

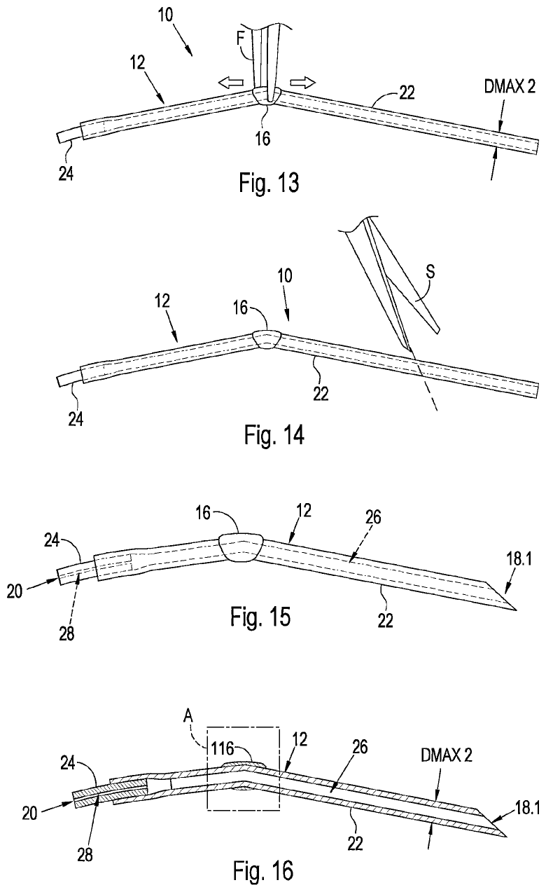

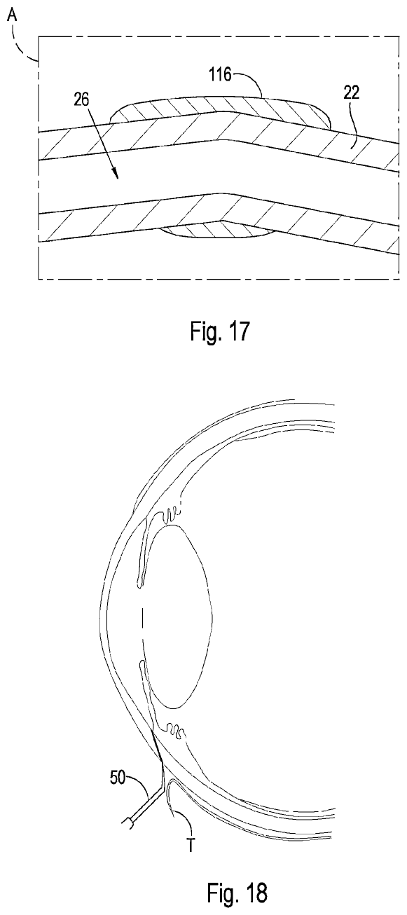

With reference to FIGS. 13-19, the manner in which the shunt 10 is implanted in the anterior chamber of an eye of a patient, is illustrated. The anatomical dimensions and ideal bleb location of the eye of the patient are initially measured by the surgeon performing a visual observation of the patient's eye in order to determine an optimal length for the shunt.

With specific reference to FIG. 13, the fixation body 16 is then slid along the distal portion of the elongate duct by a surgeon gripping the fixation body 16 using forceps and applying a force to the fixation body in a direction for displacing the fixation body along the distal portion until a desired position along the distal portion is achieved. An important consideration in this regard is the position at which the surgeon would like to form a bleb into which aqueous humor from the anterior chamber is allowed to drain after implantation of the shunt.

An internal diameter of the internal passage 30 of the fixation body is slightly less than an external diameter of the distal portion of the elongate duct, causing the fixation body to be located on the distal portion 22 of the duct 12 in an interference fit permitting sliding displacement of the fixation body when sufficient force is applied to the fixation body in order to overcome frictional forces holding the fixation body on the distal position.

With reference to FIGS. 14-17, a distal portion 22 of the elongate duct 12 of the shunt is then cut by a surgeon using surgical scissors S, so as to adjust the length of the shunt to a desired length. More specifically, an angled bevel cut is made in the distal portion so as to provide a distal end 18.1 having a sharp point for facilitating insertion of the distal portion along the scleral channel and into the anterior or posterior chamber.

With reference to FIG. 18, a portion T of the conjunctiva-tenon's complex is released from the limbus and retracted. A passageway is then created through scleral tissue by a surgeon using a surgical blade I so as to form a scleral channel from an external position having an entry point approximately 3 mm from the limbus to an exit point into the anterior chamber at the trabecular network. The distal portion of the elongate duct of the shunt is then inserted into the scleral channel until the fixation body lies within the scleral channel and the distal end of the elongate duct lies within the anterior chamber.

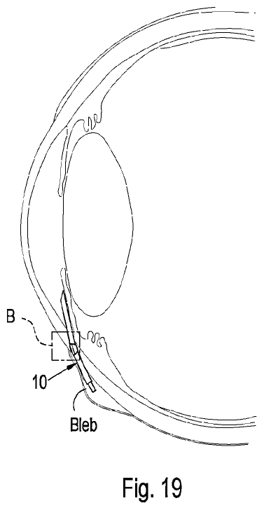

In FIG. 19, the shunt 10 is shown implanted in the anterior chamber of the patient's eye, providing a passageway along which aqueous humor is drained from the anterior chamber into a bleb formed between scleral tissue and the conjunctiva-tenon's complex near to the limbus.

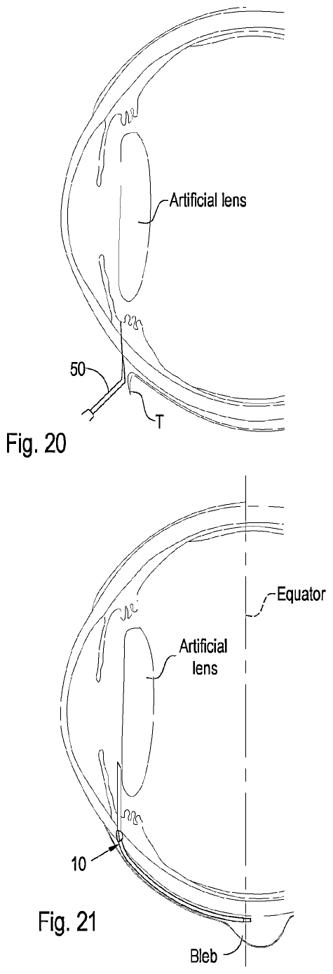

With reference to FIGS. 20 and 21, the manner in which the shunt 10 is implanted in the posterior chamber of an eye and used, is illustrated. The shunt 10 is implanted in the same manner as described above in relation to the implantation of the shunt into the anterior chamber of the eye, with the only difference being that the scleral channel is formed with an exit point into the posterior chamber at the ciliary sulcus.

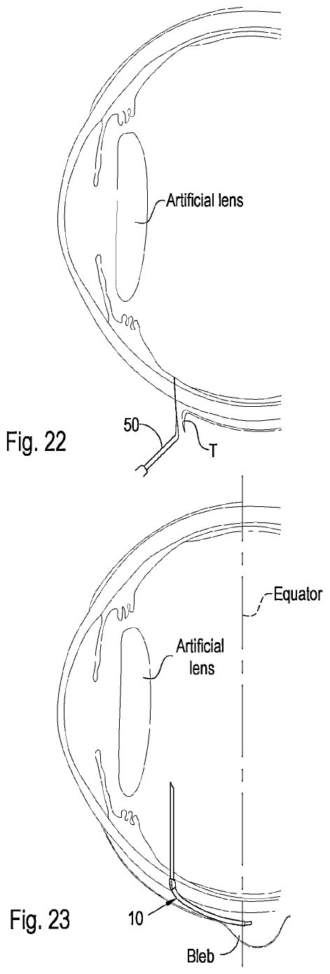

With reference to FIGS. 22 and 23, the manner in which the shunt 10 is implanted in the vitreous chamber of an eye and used, is illustrated. The shunt 10 is implanted in the same manner as described above in relation to the implantation of the shunt into the anterior chamber of the eye, with the only difference being that the scleral channel is formed in the pars plana region of the ciliary body with an exit point into the vitreous chamber.

The shunt 10 provides an extended length of deformable tube with a fixation body slidably located thereon so as to allow a surgeon to adjust the position of the fixation body along the elongate duct to thereby adjust the length of the shunt to the anatomical dimensions of the eye of a patient and provide for optimal positioning of a bleb into which aqueous humor can drain.

The proximal portion of the elongate duct provides the shunt with a capillary valve, while the distal portion of the elongate duct may be cut so as to adjust the length of the shunt without affecting the capillary valve or altering the resistance to fluid flow along the fluid passageway of the shunt significantly.

The interference fit of the fixation body on the distal portion of the shunt provides for resistance to migration of the fixation body along the elongate duct when the shunt is implanted, while allowing for movement along the distal portion in order to adjust the length of the shunt. The outwardly projecting formations furthermore create a fluid seal in the scleral channel.

In summary, a surgical method for treating glaucoma in a patient by lowering intraocular pressure in an eye of a patient, comprises:
providing the shunt 10 as defined and described hereinabove;
measuring the anatomical dimensions of the eye of the patient and a distance to a desired location for the formation of a bleb into which aqueous humor can drain, in order to determine an optimal length for the shunt;
sliding the fixation body 16 along the distal portion 22 of the elongate duct 12 until a desired fixation body position is achieved corresponding to a desired length of the distal portion of the elongate duct and a desired position of the fixation body along the elongate duct for fixing the distal portion within the scleral channel;
cutting the distal portion of the elongate duct so as to adjust a length of the elongate duct;
opening the conjunctival/tenon's complex to create a pocket between the conjunctival/tenon's complex and the sclera;
using the surgical blade 54, creating a passageway through scleral tissue so as to form a scleral channel from an external position at the pocket in the conjunctival/tenon's complex to the anterior/posterior chamber of the eye from which aqueous fluid is to be diverted;
inserting the distal portion of the elongate duct of the shunt into the scleral channel until the fixation body 16 lies within the scleral channel and the distal end 18.1 of the elongate duct lies within the anterior/posterior chamber of the eye; and
closing the conjunctival/tenon's complex leaving the proximal end of the shunt lying within the pocket.

The surgical method includes, prior to creating the passageway using the cutting blade, marking a point on the sclera in the pocket between the conjunctival/tenon's complex and the sclera at a predetermined distance from the limbus which may be approximately 3 mm from the limbus.

The surgical method may include creating the passageway through scleral tissue, from the predetermined position on the sclera to enter the chamber of the eye in a single step (as is illustrated in FIGS. 18, 20 and 22).

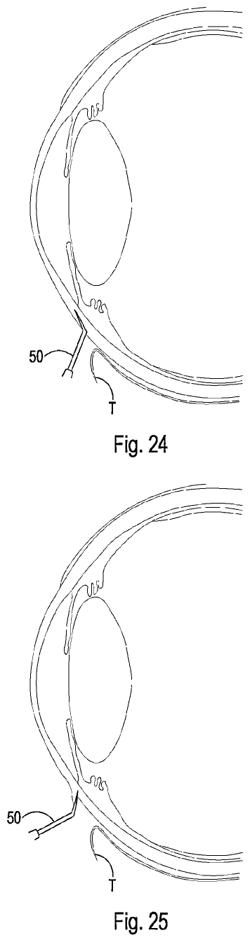

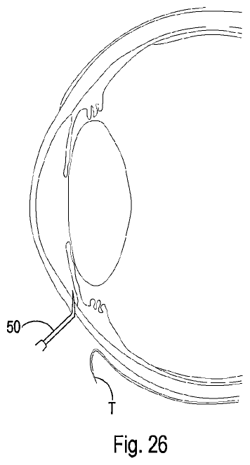

Alternatively, the surgical method may include creating the passageway through scleral tissue, from the predetermined position on the sclera to enter the chamber of the eye in three steps as follows:
1) using the cutting blade of the surgical instrument, creating a passageway through scleral tissue at an angle approximately parallel to the wall of the ocular globe so as to form a scleral channel extending from the predetermined position on the sclera to a point about 1 mm from the limbus (see FIG. 24);
2) changing the direction of the cutting blade towards the iris plane (see FIG. 25); and
3) advancing the cutting blade to enter the anterior chamber of the eye at about the region of the trabecular meshwork (see FIG. 26).

The shunt and method described hereinabove ameliorate the shortcomings of the prior art devices described above by reducing the maximal cross-sectional dimension of the fixation body to less than the maximal cross-sectional dimension of the surgical instrument used to create the scleral channel while still providing an adequate fluid seal. This reduces surgical invasiveness and the risk of device erosion and migration. The multi-bevelled and flat-bodied surgical blade of the present invention also assists a surgeon to easily create an appropriate scleral channel for the elongate duct of the shunt with a reduced risk of fluid leakage.

1. A surgical kit for treating glaucoma in a patient, the surgical kit comprising:
a surgical instrument having a flat cutting blade for creating a passageway through scleral tissue, the flat blade having a first maximal cross-sectional dimension; and
a shunt for diverting aqueous humor from a chamber of the eye of the patient, the shunt comprising a deformable elongate duct having a distal portion which is locatable in the scleral passageway and which defines a distal end of the elongate duct; and a fixation body located on the elongate duct intermediate the ends of the elongate duct, the distal portion of the elongate duct of the shunt having a second maximal cross-sectional dimension that is less than the first maximal cross-sectional dimension; and the fixation body having a third maximal cross-sectional dimension that is greater than the second maximal cross-sectional dimension and less than the first maximal cross-sectional dimension.

2. The surgical kit of claim 1, wherein the cutting blade of the surgical instrument has bevelled cutting surfaces at opposite sides of the cutting blade at a tip region of the cutting blade.

3. The surgical kit of claim 2, wherein the bevelled cutting tip has a length of approximately 2 mm.

4. The surgical kit of claim 1, wherein the chamber of the eye in which the shunt is implanted may be the anterior chamber or the posterior chamber or the vitreous chamber of the eye.

5. The surgical kit of claim 1, wherein the elongate duct of the shunt has a proximal portion defining a proximal end of the elongate duct, the proximal portion being locatable externally of the scleral passageway and having a proximal capillary lumen which is in fluid flow communication with the distal lumen, the proximal lumen having an internal diameter which is relatively smaller than an internal diameter of the distal lumen so as to reduce a flow rate of aqueous humor and regulate pressure along the proximal capillary lumen sufficient to prevent hypotony.

6. The surgical kit of claim 1, wherein the fixation body of the shunt is slidably located on the distal portion of the elongate duct for fixing the distal portion of the duct within the scleral channel at a desired position.

7. The surgical kit of claim 6, wherein the fixation body is frictionally located on the distal portion of the elongate duct in an arrangement wherein a coefficient of friction acting between the fixation body and the elongate duct is sufficient to adequately resist movement of the fixation body relative to the elongate duct when the elongate duct is implanted in the scleral passageway of the patient yet permits sliding displacement of the fixation body relative to the elongate duct when a moderate force is applied to the fixation body by a surgeon.

8. The surgical kit of claim 1, wherein the fixation body defines an internal passage within which the elongate duct is received.

9. The surgical kit of claim 1, wherein the fixation body is located on the elongate duct in an interference fit wherein an internal diameter of the fixation body is slightly less than an external diameter of the distal portion of the elongate duct.

10. The surgical kit of claim 1, wherein the fixation body has a pair of laterally-extending flanges which project outwardly from opposite sides thereof.

11. The surgical kit of claim 1, wherein the fixation body has a convexly rounded lower surface and a substantially flat upper surface.

12. The surgical kit of claim 1, wherein the fixation body is of a rigid construction.

13. The surgical kit of claim 1, wherein the fixation body defines a curved internal passage within which the elongate duct is received.

14. A surgical method for treating glaucoma in a patient, the surgical method comprising:
providing a surgical kit;
opening the conjunctival/tenon's complex to create a pocket between the conjunctival/tenon's complex and the sclera;
using the cutting blade of the surgical instrument, creating a passageway through scleral tissue so as to form a scleral channel from an external position at the pocket in the conjunctival/tenon's complex to a chamber of the eye from which aqueous fluid is to be diverted;
inserting the distal portion of the elongate duct of the shunt into the scleral channel until the distal end of the elongate duct lies within the chamber of the eye and the fixation body is located within the scleral channel for fixing and sealing the distal portion of the elongate duct within the scleral tissue at a desired position relative to the limbus; and
closing the conjunctival/tenon's complex leaving the proximal end of the shunt lying within the pocket.

15. The surgical method of claim 14, which includes creating the passageway through scleral tissue, from the predetermined position on the sclera to enter the chamber of the eye in a single step.

16. The surgical method of claim 14, which includes creating the passageway through scleral tissue, from the predetermined position on the sclera to enter the chamber of the eye comprising the steps as follows:
using the cutting blade of the surgical instrument, creating a passageway through scleral tissue at an angle approximately parallel to the wall of the ocular globe so as to form a scleral channel extending from the predetermined position on the sclera to a point about 1 mm from the limbus;
changing the direction of the cutting blade towards the iris plane; and
advancing the cutting blade to enter the anterior chamber of the eye at about the region of the trabecular meshwork.
